(19) United States
(12) Patent Application Publication
(10) Pub. No.: US 2026/0035429 A1
(43) Pub. Date: Feb. 5, 2026

(54) METHOD FOR PRODUCING DUAL FUNCTION PROTEINS AND ITS DERIVATIVES

(71) Applicant: YUHAN CORPORATION, Dongjak-gu (KR)

(72) Inventors: Byung Hyun CHOI, Suwon-si (KR); In Hwan LIM, Suwon-si (KR); Jun Young PARK, Seoul (KR); Jin Hyoung LEE, Yongin-si (KR); Ki Hong KIM, Suwon-si (KR); Hae Yong JO, Seoul (KR); Jun KIM, Seoul (KR); Moo Young SONG, Suwon-si (KR); Jong Gyun KIM, Anyang-si (KR)

(73) Assignee: YUHAN CORPORATION, Dongjak-gu (KR)

(21) Appl. No.: 19/179,601

(22) Filed: Apr. 15, 2025

Related U.S. Application Data
(63) Continuation of application No. 18/068,506, filed on Dec. 19, 2022, now abandoned.
Application No. 18/068,506 is a continuation of application No. 16/606,279, now Pat. No. 11,560,416.

(30) Foreign Application Priority Data
Apr. 21, 2017 (KR) 10-2017-0051758

(51) Int. Cl. C07K 14/605; C07K 14/50; C07K 16/46; C07K 19/00; C12N 5/00; C12N 15/00; C12P 21/02

(52) U.S. Cl. CPC C07K 14/605; C07K 14/50; C12P 21/02; C07K 16/46; C07K 19/00; C07K 2319/30; C12N 5/00; C12N 15/00

(57) ABSTRACT

A method for producing a dual function protein includes a biologically active protein and an FGF21 mutant protein. The method allows stable production of a target protein by effectively preventing decomposition of the target protein, and thus has a high potential for commercial usage.

### CROSS-REFERENCE TO RELATED APPLICATIONS

This application is a Continuation of U.S. application Ser. No. 18/068,506 filed Dec. 19, 2022, which is a Continuation of U.S. application Ser. No. 16/606,279 filed Oct. 18, 2019, which is a National Stage of International Application No. PCT/KR2018/004599, filed Apr. 20, 2018, claiming priority to Korean Patent Application No. 10-2017-0051758, filed Apr. 21, 2017, the above-noted applications incorporated herein by reference in their respective entireties.

### INCORPORATION BY REFERENCE OF SEQUENCE LISTING

The content of the electronically submitted sequence listing, file name: Q308285_Sequence_listing_as_filed.xml; size: 84,615 byte; and date of creation: Apr. 15, 2025, filed herewith, is incorporated herein by reference in its entirety.

### TECHNICAL FIELD

The present invention relates to a method for producing a dual function protein comprising a biologically active protein and a fibroblast growth factor 21 (an FGF21) mutant protein.

### BACKGROUND ART

When an animal cell is used to produce a recombinant protein, there might be a problem that a specific region of the target protein may be clipped by a protease secreted by an animal cell (host cell) to cause heterogeneity, reduction or inactivation of the recombinant protein. In addition, such clipping of the expressed protein also leads to the problem that it gets difficult to maintain “lot to lot” homogeneity during production and purification processes. For this reason, it is necessary to keep the protease at a low level or suppress the protease activity during the production of a recombinant protein.

As an alternative to solve the problem, a production method in which inhibitors against serine, cysteine, aspartic acid or aminopeptidase (such as aprotinin, bestatin, leupeptin, E-64 and pepstatin A, etc.) are added in the culture medium was proposed (see WO 1990-002175, EP 0,306,968, and U.S. Pat. No. 5,851,800). However, the use of these inhibitors in commercial production is not effective because of cytotoxicity and the need for extra efforts to prove that they have been completely removed from the final product.

In addition, among conventional alternatives, a universal method applicable to all target proteins produced in host cells has not been found yet.

### DISCLOSURE OF INVENTION

### Technical Problem

It is an object of the present invention to provide a culture method for producing a dual function protein comprising a biologically active protein and an FGF21 mutant protein, which has improved pharmacokinetic parameters, high stability, less potential for aggregation to form a complex, and less immunogenic potential.

### Solution to Problem

In accordance with one object of the present invention, there is provided a method for producing a recombinant dual function protein from a mammalian host cell transformed with an expression vector containing cDNA encoding a dual function protein or a derivative thereof, the method comprising culturing the mammalian host cell in a culture medium supplemented with dextran sulfate, wherein the dual function protein comprises a fibroblast growth factor 21 (FGF21) mutant protein; a biologically active protein, or a mutant or fragment thereof; and an Fc region of an immunoglobulin, wherein the FGF21 mutant protein comprises at least one mutation selected from the group consisting of the mutations (1) to (7) below:
(1) a substitution of amino acids at positions 98 to 101 from the N-terminus of a wild-type FGF21 protein with an amino acid sequence of EIRP (SEQ ID NO: 53);
(2) a substitution of amino acids at positions 170 to 174 from the N-terminus of a wild-type FGF21 protein with an amino acid sequence of TGLEAV (SEQ ID NO: 54);
(3) a substitution of amino acids at positions 170 to 174 from the N-terminus of a wild-type FGF21 protein with an amino acid sequence of TGLEAN (SEQ ID NO: 55);
(4) a substitution of an amino acid at position 170 from the N-terminus of a wild-type FGF21 protein with an amino acid N;
(5) a substitution of an amino acid at position 174 from the N-terminus of a wild-type FGF21 protein with an amino acid N;
(6) a substitution of an amino acid at position 180 from the N-terminus of a wild-type FGF21 protein with an amino acid E, along with one or more mutations (1) to (5) above; and
(7) a mutation of 1 to 10 amino acids for reducing immunogenicity of a wild-type FGF21 protein.

### Advantageous Effects of Invention

The production method of the present invention allows stable production of a target protein by effectively preventing decomposition of the target protein.

### BRIEF DESCRIPTION OF DRAWINGS

FIG. 1 is a schematic diagram showing the result of analysis of the culture supernatant by SDS-PAGE after suspension culture of a cell line expressing a dual function protein. It was found that as the culture time elapsed, other proteins smaller than the non-clipped dual function protein were expressed together.

FIG. 2 is a schematic diagram showing the result of analysis of the culture supernatant by SDS-PAGE according to storage temperature conditions after a certain time period. The culture supernatant stored at 4° C. or −20° C. showed reduced clipping of the dual function proteins as compared to that stored at 37° C. This result indicated that the clipping phenomenon of the dual function proteins was induced by the protease derived from the host cell.

FIG. 3 is a schematic diagram showing the result of analysis by SDS-PAGE after adding protease inhibitors to the culture supernatants and storing the mixtures for a certain time period at 37° C. It was found that mainly the addition of serine protease inhibitor decreased the clipping phenomenon of the dual function protein. This result indicated that the clipping phenomenon of the dual function protein was induced by the protease derived from the host cell.

FIG. 4 is the result of analysis of the culture supernatant by SDS-PAGE after a cell culture in which dextran sulfate was added to the culture medium, and the graph thereof. The effect of reducing the clipping phenomenon of the dual function protein was not observed when dextran sulfate having a weight average molecular weight of 1.6 kDa was added, whereas clipping phenomenon of the dual function protein was reduced when 500 kDa dextran sulfate was added.

FIG. 5 is a schematic diagram showing the result of analysis of the culture supernatant by SDS-PAGE after culturing with dextran sulfates having different weight average molecular weights added to the culture medium. When dextran sulfate having a weight average molecular weight of 200 kDa or more was added to the culture medium, the clipping phenomenon of the dual function protein was reduced.

FIG. 6 is the result of SDS-PAGE analysis of the culture supernatant after the culture in which dextran sulfates at various concentrations were added to the culture medium, and the graph thereof. When dextran sulfate was added at 200-1,000 mg/L, the clipping phenomenon of the dual function protein was reduced. In addition, when the culture temperature was changed to 32° C. during the culture, the clipping phenomenon of the dual function protein could be prevented more effectively.

### BEST MODE FOR CARRYING OUT THE INVENTION

In accordance with one object of the present invention, there is provided a method for producing a recombinant dual function protein from a mammalian host cell transformed with an expression vector containing cDNA encoding a dual function protein or a derivative thereof, the method comprising culturing the mammalian host cell in a culture medium supplemented with dextran sulfate, wherein the dual function protein comprises a fibroblast growth factor 21 (FGF21) mutant protein; a biologically active protein, or a mutant or fragment thereof; and an Fc region of an immunoglobulin, wherein the FGF21 mutant protein comprises at least one mutation selected from the group consisting of the mutations (1) to (7) below:
(1) a substitution of amino acids at positions 98 to 101 from the N-terminus of a wild-type FGF21 protein with an amino acid sequence of EIRP (SEQ ID NO: 53) (hereinafter, “EIRP”);
(2) a substitution of amino acids at positions 170 to 174 from the N-terminus of a wild-type FGF21 protein with an amino acid sequence of TGLEAV (SEQ ID NO: 54) (hereinafter, “TGLEAV”);
(3) a substitution of amino acids at positions 170 to 174 from the N-terminus of a wild-type FGF21 protein with an amino acid sequence of TGLEAN (SEQ ID NO: 55) (hereinafter, “TGLEAN”);
(4) a substitution of an amino acid at position 170 from the N-terminus of a wild-type FGF21 protein with an amino acid N (hereinafter, “G170N”);
(5) a substitution of an amino acid at position 174 from the N-terminus of a wild-type FGF21 protein with an amino acid N (hereinafter, “G174N”);
(6) a substitution of an amino acid at position 180 from the N-terminus of a wild-type FGF21 protein with an amino acid E, along with one or more mutations (1) to (5) above (hereinafter, “A180E”); and
(7) a mutation of 1 to 10 amino acids for reducing immunogenicity of a wild-type FGF21 protein.

### FGF21 Mutant Protein

The wild-type FGF21 protein, a hormone known to play an important role in glucose and lipid homeostasis, may be one derived from mammals such as humans, mice, pigs, monkeys, etc., preferably from humans. More preferably, the wild-type FGF21 protein may be the wild-type human FGF21 protein having an amino acid sequence represented by SEQ ID NO: 1.

Preferably, the mutation included in the FGF21 mutant proteins may be any one of the mutations of EIRP(SEQ ID NO: 53), TGLEAV(SEQ ID NO: 54), TGLEAN(SEQ ID NO: 55), G170N and G174N; a combination of any one of the mutations of TGLEAV(SEQ ID NO: 54), TGLEAN(SEQ ID NO: 55), G170N and G174N and the mutation of EIRP(SEQ ID NO: 53); a combination of any one of the mutations of EIRP(SEQ ID NO: 53), TGLEAV(SEQ ID NO: 54), TGLEAN(SEQ ID NO: 55), G170N and G174N and the mutation of A180E; or a combination of any one of the mutations of TGLEAV(SEQ ID NO: 54), TGLEAN(SEQ ID NO: 55), G170N and G174N, the mutation of EIRP and the mutation of A180E.

The EIRP(SEQ ID NO: 53) refers to a mutation in which LLLE(SEQ ID NO: 57), the amino acids at positions 98 to 101 from the N terminus of a wild-type FGF21 protein, is substituted with EIRP(SEQ ID NO: 53). Further, the TGLEAV(SEQ ID NO: 54) refers to a mutation in which GPSQG(SEQ ID NO: 58), the amino acids at positions 170 to 174 from the N terminus of a wild-type FGF21 protein, is substituted with TGLEAV(SEQ ID NO: 54). In addition, the TGLEAN(SEQ ID NO: 55) refers to a mutation in which GPSQG(SEQ ID NO: 58), the amino acids at positions 170 to 174 from the N terminus of a wild-type FGF21 protein, is substituted with TGLEAN(SEQ ID NO: 55). Further, the G170N refers to a mutation in which G, the amino acid at position 170 from the N terminus of a wild-type FGF21 protein, is substituted with N. In addition, the G174N refers to a mutation in which G, the amino acid at position 174 from the N terminus of a wild-type FGF21 protein, is substituted with N.

Furthermore, the FGF21 mutant proteins may have a conformation, in which 1 to 10 amino acids at the N-terminus or C-terminus is (are) deleted as compared to the wild-type FGF21 protein. More preferably, the FGF21 mutant proteins may include an amino acid sequence represented by any one of SEQ ID NOs: 6 to 23. Still more preferably, the FGF2l mutant proteins may include an amino acid sequence represented by any one of SEQ ID NOs: 6 to 23 and further have a conformation, in which 1 to 10 amino acids at the N-terminus or C-terminus is (are) deleted as compared to the wild-type FGF21 protein.

In the dual function protein, an amino acid residue N of FGF21 mutant protein introduced by a mutation may be glycosylated.

### Biologically Active Protein

The biologically active protein may be one selected from the group consisting of insulin, C-peptide, leptin, glucagon, gastrin, gastric inhibitory polypeptide (GIP), amylin, calcitonin, cholecystokinin, peptide YY, neuropeptide Y, bone morphogenetic protein-6 (BMP-6), bone morphogenetic protein-9 (BMP-9), oxyntomodulin, oxytocin, glucagon-like peptide-1 (GLP-1), glucagon-like peptide-2 (GLP-2), irisin, fibronectin type III domain-containing protein 5 (FNDC5), apelin, adiponectin, C1q and tumor necrosis factor related protein (CTRP family), resistin, visfatin, omentin, retinol binding protein-4 (RBP-4), glicentin, angiopoietin, interleukin-22 (IL-22), exendin-4 and growth hormone. Preferably, the biologically active protein may be one selected from GLP-1, a mutant thereof and exendin-4.

The GLP-1 protein is an incretin hormone consisting of 31 amino acids, which is secreted by L cells in the intestinal tract stimulated by food, etc. For example, the GLP-1 protein may be represented by the amino acid sequence of SEQ ID NO: 29.

A mutant of GLP-1 may be represented, for example, by the amino acid sequence of any one of SEQ ID NOs: 30 to 33.

### Fc Region of Immunoglobulin

As used herein, the term “Fc region,” “Fc fragment,” or “Fc” refers to a protein, which includes a heavy chain constant region 1 (CH1), a heavy chain constant region 2 (CH2) and a heavy chain constant region 3 (CR3) of an immunoglobulin, but does not include variable regions of the heavy and light chains and a light chain constant region 1 (CLI) of an immunoglobulin. Additionally, as used herein, the term “Fe region mutant” refers to one prepared by substituting part of amino acid(s) of an Fe region or by combining Fe regions of different types.

The Fc region of immunoglobulin may be an entire Fe region constituting an antibody, a fragment thereof, or an Fe region mutant. Additionally, the Fe region includes a molecule in the form of a monomer or multimer, and may further include a hinge region of the heavy chain constant region. The Fe region mutant may be modified to prevent cleavage at the hinge region. Furthermore, the hinge sequence of the Fe may have a substitution in some amino acid sequences to reduce antibody-dependent cell-mediated cytotoxicity (ADCC) or complement-dependent cytotoxicity (CDC). In addition, part of the amino acid sequence of the Fe hinge sequence may be substituted to inhibit the rearrangement of the Fab region. A lysine residue at the C-terminus of the Fe may be removed.

Preferably, the Fe region of immunoglobulin may be any one of IgG1, IgG2, IgG3, IgG4 and IgD Fe regions; or a hybrid Fe, which is a combination thereof. Further, the hybrid Fe may include an IgG4 region and an IgD region. Further, the hybrid Fc region may include part of the hinge sequence and CH2 of an IgD Fe, and CH2 and CH3 sequences of IgG4 Fe.

In addition, the Fe fragment of the present invention may be in the form of wild-type glycosylated chain, more glycosylated chain than the wild-type, less glycosylated chain than the wild-type, or deglycosylated chain. The increase, decrease, or removal of glycosylated chain may be performed by a conventional method known in the art, such as a chemical method, an enzymnatic method, and a genetic engineering method using microorganisms.

Preferably, the immunoglobulin Fc region may be represented by an amino acid sequence selected from SEQ ID NOs: 24 to 28.

### Dual Function Protein

The dual function protein may include a biologically active protein, an Fc region of an immunoglobulin and an FGF21 mutant protein, linked in this order from the N-terminus to the C-terminus. Further, the dual function protein may include an FGF21 mutant protein, an Fc region of an immunoglobulin and a biologically active protein, linked in this order from the N-terminus to the C-terminus. Preferably, the dual function protein may include a GLP-1 mutant protein, an Fc region of an immunoglobulin and an FGF21 mutant protein, linked in this order from the N-terminus to the C-terminus. Further, the dual function protein may include an FGF21 mutant protein, an Fe region of an immunoglobulin and a GLP-1 mutant protein, linked in this order from the N-terminus to the C-terminus.

### Linker

Additionally, the dual function protein may further include a linker.

The dual function protein may be in the form, in which the FGF21 mutant protein is directly connected to the N-terminus or C-terminus of the immunoglobulin Fe region, or the FGF21 mutant protein is connected to the immunoglobulin Fc region via a linker.

In such case, the linker may be connected to the N-terminus, C-terminus, or a free radical of the Fc fragment, and also, may be connected to the N-terminus, C-terminus, or a free radical of the FGF21 mutant protein. When the linker is a peptide linker, the connection may occur in any region. For example, the linker may be connected to the C-terminus of the immunoglobulin Fe region and the N-terminus of the FGF21 mutant protein to form a fusion protein of the immunoglobulin Fe region and the FGF21 mutant protein. Furthermore, the dual function protein of the present invention may be in the form, in which a biologically active protein is linked to the N-terminus of the Fc region of immunoglobulin of the fusion protein.

When the linker and Fe are separately expressed and then connected, the linker may be a crosslinking agent known in the art. Examples of the crosslinking agent may include 1,1-bis(diazoacetyl)-2-phenylethane, glutaraldehyde, imidoesters including N-hydroxysuccinimide ester such as 4-azidosalicylic acid and disuccinimidyl esters such as 3,3-dithiobis(succinimidylpropionate), and bifunctional maleimides such as bis-N-maleimido-1,8-octane, but are not limited thereto.

Further, the linker may be a peptide. Preferably, the linker may be a peptide consisting of 10 to 30 amino acid residues.

Furthermore, alanine may additionally be attached to the end of linker. Preferably, the linker may be a peptide having an amino acid sequence represented by any one of SEQ ID NOs: 2 to 5.

The dual function protein may be in a form in which a dimer or multimer of FGF21 mutant proteins, in which one or more FGF21 mutant proteins linked together, is connected to an immunoglobulin Fc region. Additionally, the dual function protein may be in a form of a dimer or multimer in which two or more immunoglobulin Fc regions are linked, wherein the immunoglobulin Fc regions have the FGF21 mutant protein connected thereto.

### Mammalian Host Cell

The mammalian host cell may be any animal cell capable of expressing a recombinant dual function protein, preferably an animal cell which allows easy isolation of a targeted transformed cell. Specifically, the mammalian host cells may be immortal hybridoma cells, NS/0 myeloma cells, 293 cells, Chinese hamster ovary cells (CHO cells), HeLa cells, CAP cells (human amniotic fluid-derived cells), or COS cells.

### Dextran Sulfate

As a result of applying the protease inhibitor to the cell culture of a dual function protein of the present invention, the effect of preventing the clipping phenomenon of the dual function protein by the protease derived from the host cell was insufficient.

The dextran sulfate may have a weight average molecular weight of 20 to 5,000 kDa. Specifically, the dextran sulfate may have a weight average molecular weight of 200 to 5,000 kDa.

In addition, the culture medium may contain the dextran sulfate at a concentration of 0.01 to 10 g/L. Specifically, the culture medium may contain the dextran sulfate at a concentration of 0.1 to 10 g/L, or 0.1 to 1 g/L.

### Culture

The culturing may comprise a step for primary-culturing the mammalian host cell at 34 to 37° C. in a culture medium supplemented with dextran sulfate; and a step for secondary-culturing the primary-cultured medium at 28 to 33° C. Specifically, the primary-culturing may be conducted for 24 to 144 hours. Also, the secondary-culturing may be conducted at 31 to 33° C.

The dual function protein is a polypeptide in which GLP-1 and FGF21 variants, biologically active proteins, are fused to the Fc region of an immunoglobulin, and is expressed in an intact form when produced by animal cell culture, and shows activity as a composition for preventing or treating hepatitis, hepatic fibrosis, and hepatic cirrhosis.

### MODE FOR THE INVENTION

Hereinafter, exemplary embodiments of the present invention will be described in detail with reference to the examples. However, these examples according to the present invention can be modified in many different forms and the scope of the present invention should not be construed as limited to the examples set forth herein.

### EXAMPLES

### Preparation Example 1. Preparation of Host Cells for Expression of Dual Function Proteins

### 1-1: Preparation of Expression Vectors for Expression of Dual Function Proteins

The position, sequence information, target and expected effect of each mutation introduced into the FGF21 protein are listed in Table 1 below (in Table 1, N refers to glycosylated asparagine (N)). Further, FGF21 mutant proteins including the mutations described in Table 1 are listed in Table 2 below.

TABLE 1

| | | Original | Mutated | | |
|---|---|---|---|---|---|
| Sequence | Position | sequence | sequence | Target | Expected effect |
| EIRP | 98-101 | LLLE | EIRP | Substitution with FGF19 | Improvement of |
| (SEQ ID | | (SEQ ID | (SEQ ID | sequence | stability and |
| NO: 53) | | NO: 57) | NO: 53) | | pharmacokinetics |
| TGLEAV | 170-174 | GPSQG | TGLEAV | Substitution with FGF19 | Improvement of |
| (SEQ ID | | (SEQ ID | (SEQ ID | sequence | pharmacokinetics |
| NO: 54) | | NO: 58) | NO: 54) | | |
| TGLEAN | 170-174 | GPSQG | TGLEAN | Substitution with FGF19 | Improvement of |
| (SEQ ID | | (SEQ ID | (SEQ ID | sequence, and | pharmacokinetics |
| NO: 55) | | NO: 58) | NO: 55) | addition of N-glycosylation | |
| G170N | 170 | G | N | Point mutation, and addition | Improvement of |
| | | | | of N-glycosylation | pharmacokinetics |
| G174N | 174 | G | N | Point mutation, and addition | Improvement of |
| | | | | of N-glycosylation | pharmacokinetics |
| A180E | 180 | A | E | Point mutation | Improvement of |
| | | | | | pharmacokinetics |

TABLE 2

| SEQ ID NO | Sequence of FGF21 mutant protein |
|---|---|
| 6 | FGF21 (EIRP) |
| 7 | FGF21 (TGLEAV) |
| 8 | FGF21 (TGLEAN) |
| 9 | FGF21 (G170N) |
| 10 | FGF21 (G174N) |
| 11 | FGF21 (EIRP, TGLEAV) |
| 12 | FGF21 (EIRP, TGLEAN) |
| 13 | FGF21 (EIRP, G170N) |
| 14 | FGF21 (EIRP, G174N) |
| 15 | FGF21 (EIRP, A180E) |
| 16 | FGF21 (TGLEAV, A180E) |
| 17 | FGF21 (TGLEAN, A180E) |
| 18 | FGF21 (G170N, A180E) |
| 19 | FGF21 (G174N, A180E) |
| 20 | FGF21 (EIRP, TGLEAV, A180E) |
| 21 | FGF21 (EIRP, TGLEAN, A180E) |
| 22 | FGF21 (EIRP, G170N, A180E) |
| 23 | FGF21 (EIRP, G174N, A180E) |

The GLP-1 mutant protein sequences are shown in Table 3 below, and the Fc fusion GLP-1 mutant protein sequences are shown in Table 4.

TABLE 3

| SEQ ID NO | Sequence of GLP-1 mutant protein |
|---|---|
| 30 | GLP-1 (A2G) |
| 31 | GLP-1 (GE) |
| 32 | GLP-1 (GG) |
| 33 | GLP-1 (GEG) |

TABLE 4

| SEQ ID NO | Fc fusion GLP-1 mutant protein |
|---|---|
| 34 | DFD52: GLP1(A2G)-HyFc5 |
| 35 | DFD53: GLP1(A2G)-HyFc40 |
| 36 | DFD54: GLP1(GE)-HyFc5 |
| 37 | DFD55: GLP1(GE)-HyFc40 |
| 38 | DFD56: GLP1(GG)-HyFc5 |
| 39 | DFD57: GLP1(GG)-HyFc40 |
| 40 | DFD58: GLP1(GEG)-HyFc5 |
| 41 | DFD59: GL1(GEG)-HyFc40 |

In Table 4, HyFc5 represents SEQ ID NO: 27 and HyFc40 represents SEQ ID NO: 28.

Further, the sequences of the dual function proteins including the GLP-1 mutant proteins and FGF21 mutant proteins are listed in Table 5 below. Each dual function protein contains a GLP-1 mutant protein, an Fe region of an immunoglobulin, a linker and an FGF21 mutant protein connected in this order from the N-terminus to C-terminus.

TABLE 5

| | | Sequence of | | | |
|---|---|---|---|---|---|
| SEQ | Material | GLP-1 mutant | | | Changes in FGF21 |
| ID NO | code | protein | Fusion carrier | Linker sequence | sequence |
| 43 | DFD23 | GLP-1 (A2G) | hyFc40 | GS3 | FGF21 |
| | | | (SEQ ID NO: 28) | (SEQ ID NO: 4) | (EIRP, TGLEAV) |
| 44 | DFD24 | GLP-1 (GE) | hyFc5 | GS3 | FGF21 |
| | | | (SEQ ID NO: 27) | (SEQ ID NO: 4) | (EIRP, TGLEAV) |
| 45 | DFD25 | GLP-1 (GE) | hyFc40 | GS3 | FGF21 |
| | | | (SEQ ID NO: 28) | (SEQ ID NO: 4) | (EIRP, TGLEAV) |
| 46 | DFD26 | GLP-1 (GG) | hyFc5 | GS3 | FGF21 |
| | | | (SEQ ID NO: 27) | (SEQ ID NO: 4) | (EIRP, TGLEAV) |
| 47 | DFD27 | GLP-1 (GG) | hyFc40 | GS3 | FGF21 |
| | | | (SEQ ID NO: 28) | (SEQ ID NO: 4) | (EIRP, TGLEAV) |
| 48 | DFD28 | GLP-1 (GEG) | hyFc5 | GS3 | FGF21 |
| | | | (SEQ ID NO: 27) | (SEQ ID NO: 4) | (EIRP, TGLEAV) |
| 49 | DFD29 | GLP-1 (GEG) | hyFc40 | GS3 | FGF21 |
| | | | (SEQ ID NO: 28) | (SEQ ID NO: 4) | (EIRP, TGLEAV) |
| 50 | DFD69 | GLP-1 (GEG) | hyFc40 | GS3 | FGF21 (EIRP, |
| | | | (SEQ ID NO: 28) | (SEQ ID NO: 4) | TGLEAV, A180E) |
| 51 | DFD112 | GLP-1 (GEG) | hyFc40 | GS3 | FGF21 (EIRP, |
| | | | (SEQ ID NO: 28) | (SEQ ID NO: 4) | TGLEAN, A180E) |
| 52 | DFD114 | GLP-1 (GEG) | hyFc40 | GS3 | FGF21 (EIRP, |
| | | | (SEQ ID NO: 28) | (SEQ ID NO: 4) | G170N, A180E) |

Specifically, the nucleotide sequences encoding each of the dual function proteins were synthesized after consulting with Bioneer Corporation (Korea) based on the amino acid sequence of each protein. NheI and NotI restriction enzyme sequences were added to the 5′ terminus and 3′ terminus of the nucleotide sequences encoding each of the dual function proteins and an initiation codon for protein translation and a leader sequence (SEQ ID NO: 56, MDAMLRGLCCVLLLCGAVFVSPSHA) enabling secretion of the expressed protein to the outside of a cell were inserted next to the restriction enzyme sequence at the 5′ terminus. A termination codon was inserted next to the nucleotide sequence, which encodes each of the FGF21 mutant proteins. The nucleotide sequence encoding each of the dual function proteins was cloned into a pTrans-emnpty expression vector by using the two restriction enzymes NheI and NotI. The pTrans-empty expression vector, which has a CMV promoter, a pUC-derived replication origin, an SV40-derived replication origin and an ampicillin-resistance gene, was purchased from CEVEC Pharmaceuticals (Germany).

### 1-2: Construction of Plasmid DNA for Expression of Dual Function Proteins

E. coli was transformed with each of the expression vectors constructed in Preparation Example 1-1 to obtain a large quantity of plasmid DNA to be used for expression. E. coli cells, with cell walls weakened through heat shock, were transformed with each expression vector, and the transformants were plated out on an LB plate to obtain colonies. The colonies thus obtained were inoculated into LB media, cultured at 37° C. for 16 hours, and each E. coli culture containing each expression vector was obtained in a volume of 100 mL. The E. coli thereafter obtained was centrifuged to remove the culture medium, and then P1, P2, P3 solutions (QIAGEN, Cat No.: 12963) were added to break the cell walls, thereby obtaining a DNA suspension in which proteins and DNA were separated. Plasmid DNA was purified from the DNA suspension thus obtained by using a Qiagen DNA purification column. The eluted plasmid DNA was identified by agarose gel electrophoresis, and the concentrations and purities were measured using a nanodrop device (Thermo Scientific, Nanodrop Lite). The DNA thus obtained was used for expression.

### 1-3: Production of Transformed Host Cells for Expression of Dual Function Proteins

CHO DG44 cells (Chinese hamster ovary cells) were transformed with each plasmid DNA isolated in Preparation Example 1-2 using FreeStyleMAX (Invitrogen, Cat. No. 16447-100). The transformed Chinese hamster ovary cells were inoculated into a medium (CD OptiCHO, Gibco, Cat. No. 12681-011), and cultured in an incubator under the condition of 8% CO₂ and 37° C., to select and culture surviving cells with passages repeatedly. The selected cells were finally selected as a single clone by limiting dilution in a 96-well plate.

### Experimental Example 1. Suspension Culture for Expression of Dual Function Proteins, and Decomposition Phenomenon

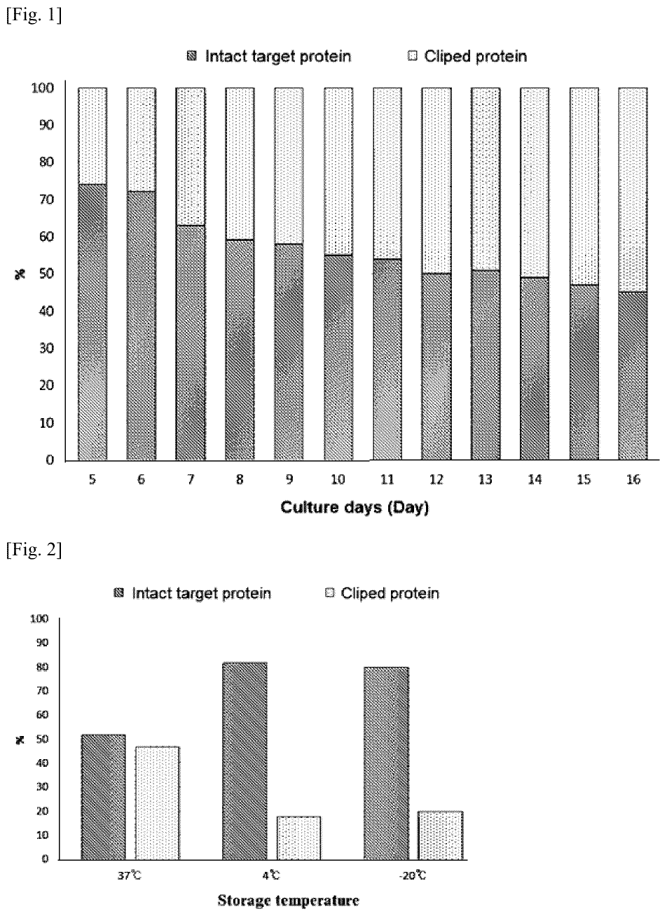

The CHO cell line transformed with the material code DFD112 (SEQ ID NO: 51) of Preparation Example 1-3 was suspension-cultured in CD OptiCHO medium supplemented with 8 mM GlutaMAX (working volume 30 ml/125 ml flask, 37° C., 8% CO₂, 120 rpm). Thereafter, the culture supernatants were stored at three different storage temperatures (37° C., 4° C., or −20° C.) for 3 days, and then the degrees of proteolysis phenomenon were evaluated by SDS-PAGE (4-12% Bis-Tris, non-reducing condition) analysis of the culture supernatant. The results of SDS-PAGE analysis are shown in FIGS. 1 and 2.

As shown in FIG. 1, it was found that proteins (85 to 110 kDa) smaller than target proteins were expressed along with the target proteins which were unclipped (intact) during cell culture for producing dual function proteins.

As shown in FIG. 2, the culture supernatants stored at 4° C. and −20° C. had less small-sized proteins in which the dual function proteins were clipped as compared to the culture supernatant stored at 37° C. Accordingly, it was found that the decomposition phenomenon of the dual function protein was caused by the proteases secreted from the host cell present in the culture supernatant.

### Experimental Example 2. Detection of Protease Inhibitors Involved in Dual Function Protein Decomposition

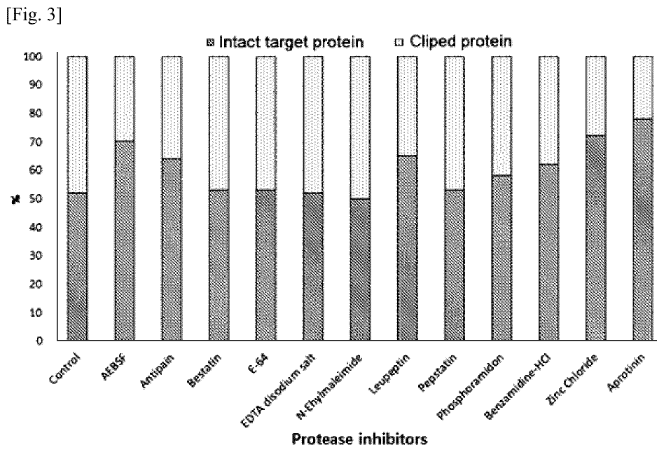

In order to examine the category of the proteases involved in the decomposition of the dual function proteins identified in Experimental Example 1, the culture supernatants of Experimental Example 1 was added with various protease inhibitors, and treated for 3 days at 37° C., which were then subjected to SDS-PAGE analysis. The protease inhibitors used herein are shown in Table 6, and SDS-PAGE analysis results are shown in FIG. 3.

TABLE 6

| | Treatment | |
|---|---|---|
| Protease inhibitors | concentration | |
| Culture supernatant (control) | N/A | |
| 4-(2-Aminoethyl)benzenesulfonyl | 1 | mM |
| fluoride (AEBSF) | | |
| Antipain | 0.1 | mM |
| Bestatin | 0.04 | mM |
| E-64 | 0.01 | mM |
| EDTA disodium salt | 1 | mM |
| N-Ethylmaleimide | 1 | mM |
| Leupeptin | 0.1 | mM |
| Pepstatin | 1.46 | mM |
| Phosphoramidon | 0.01 | mM |
| Benzamidine-HCl | 4 | mM |
| ZnCl₂ | 10 | mM |
| Aprotinin | 0.0008 | mM |

As shown in FIG. 3, it was found that the decomposition phenomenon of the dual function protein was reduced in the culture supernatants treated with proteases inhibitors related to serine protease such as AEBSF, Antipain, Leupeptin, Benzamidine-HCl and Aprotin. Accordingly, it was found that the decomposition phenomenon of the dual function protein was caused by the proteases derived from the host cell.

### Example 1. Dextran Sulfate Treatment

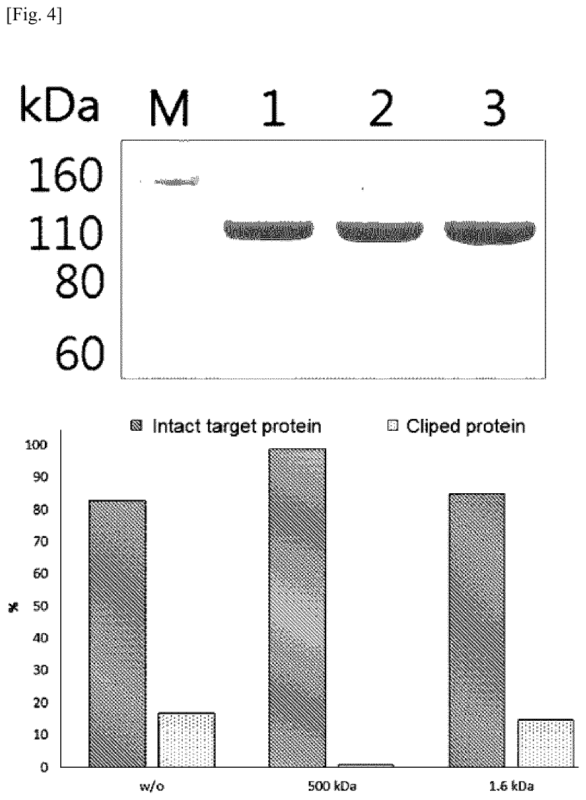

In order to inhibit the clipping phenomenon occurring during cell culture of the dual function proteins, the transformed CHO cell line of Preparation Example 1-3 was suspension-cultured in CD Dynamis medium (Gibco, cat. No. A2661501) supplemented with 6 mM glutamine for 7 days (working volume 30 mL/125 mL flask, 37° C., 8% CO₂, 120 rpm). As for the suspension culture, dextran sulfate (weight average molecular weight: 1.6 kDa or 500 kDa) was added to the culture medium at a concentration of 200 mg/L, and the culture was conducted at 32° C. by a low temperature-conversion and fed-batch culture method. Thereafter, the culture supernatant was analyzed by SDS-PAGE (4-12% Bis-Tris, non-reducing condition), and the result of the SDS-PAGE analysis and a schematic diagram thereof are shown in FIG. 4. In FIG. 4, w/o and Lane 1 are controls, 500 kDa and Lane 2 are the culture supernatants treated with 500 kDa dextran sulfate, and 1.6 kDa and Lane 3 are the culture supernatants treated with 1.6 kDa dextran sulfate.

As shown in FIG. 4, it was found that the clipping phenomenon of the dual function protein was effectively inhibited when dextran sulfate having a weight average molecular weight of 500 kDa was added to the culture medium.

### Example 2. Effect of Dextran Sulfate According to Molecular Weight

The effective concentration range of dextran sulfate to be added, whose protective effect against the clipping phenomenon of the dual function protein during cell culture was identified in Example 1, was examined.

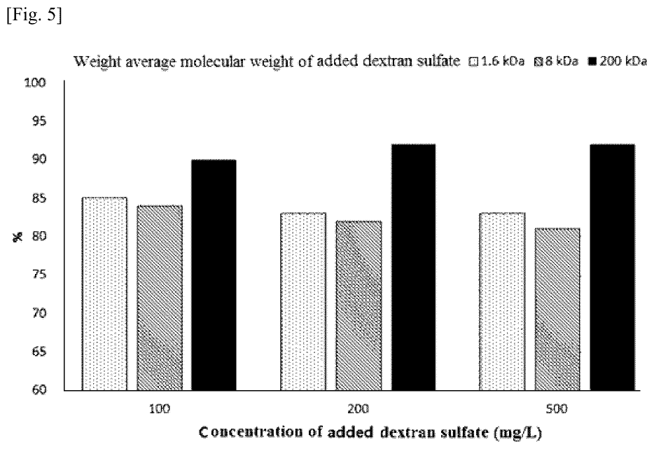

Specifically, the culture was conducted under the same condition as Example 1 except for adjusting the weight average molecular weight (1.6 kDa, 8 kDa or 200 kDa) and the concentration (100 mg/L, 200 mg/L or 500 mg/L) of the added dextran sulfate. Thereafter, the culture supernatant was analyzed by SDS-PAGE (4-12% Bis-Tris, non-reducing condition), and the result of SDS-PAGE analysis and its graph are shown in FIG. 5.

As shown in FIG. 5, the clipping phenomenon of the dual function protein was significantly reduced when the dextran sulfate having a molecular weight of 200 kDa or more was added to the culture medium at a concentration of 100 to 500 mg/L.

### Example 3. Evaluation of Culture Conditions for Prevention of Dual Function Protein Decomposition

The culture conditions for maximizing the effect of preventing the clipping of dual function proteins by dextran sulfate identified in Examples 1 and 2 were examined.

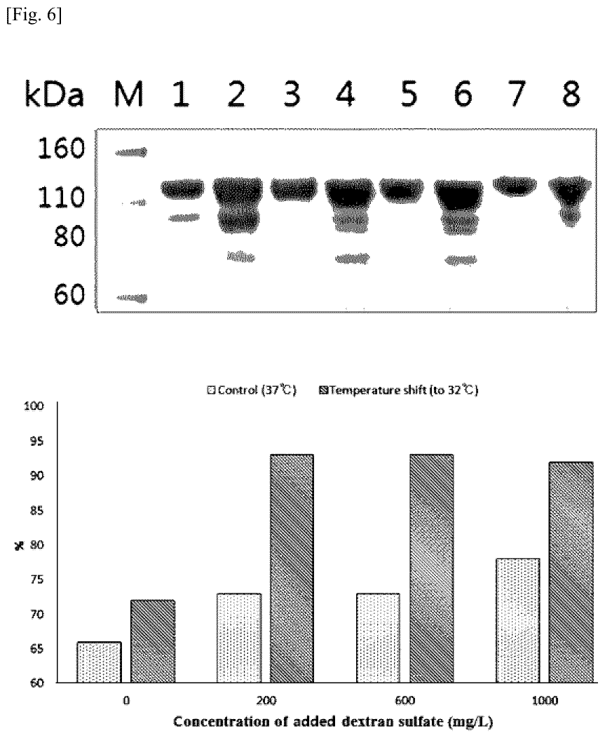

Specifically, the culture was conducted under the same condition as Example 1 except that dextran sulfate of 500 kDa was added at a concentration of 0 mg/L to 1,000 mg/L. Herein, the experiment group in which the culture temperature was changed to 32° C. on Day 4 of culture was included (see Table 7 below). Then, the culture supernatant was analyzed by SDS-PAGE (4-12% Bis-Tris, non-reducing condition), and the result of SDS-PAGE analysis and its graph are shown in FIG. 6.

TABLE 7

| | Concentration of the added | |
|---|---|---|
| Lane | Dextran sulfate | Culture condition |
| 1 | 0 | Culture at 32° C. for 3 days after |
| | | culture at 37° C. for 4 days |
| 2 | | Culture at 37° C. for 7 days |
| 3 | 200 mg/L | Culture at 32° C. for 3 days after |
| | | culture at 37° C. for 4 days |
| 4 | | Culture at 37° C. for 7 days |
| 5 | 600 mg/L | Culture at 32° C. for 3 days after |
| | | culture at 37° C. for 4 days |
| 6 | | Culture at 37° C. for 7 days |
| 7 | 1,000 mg/L | Culture at 32° C. for 3 days after |
| | | culture at 37° C. for 4 days |
| 8 | | Culture at 37° C. for 7 days |

As shown in FIG. 6, it was found that the culture supernatants of the culture with the treatment of dextran sulfate having a weight average molecular weight of 500 kDa showed significantly reduced clipping phenomenon of the dual function protein as compared to the culture supernatants of the culture without the treatment of dextran sulfate. Also, it was found that when the temperature was changed during the culture, the clipping phenomenon of the dual function protein was more effectively inhibited.

1. A method for producing a recombinant dual function protein from a mammalian host cell transformed with an expression vector containing cDNA encoding a dual function protein or a derivative thereof, the method comprising culturing the mammalian host cell in a culture medium supplemented with dextran sulfate, wherein the dual function protein comprises a fibroblast growth factor 21 (FGF21) mutant protein; a biologically active protein, or a mutant or fragment thereof; and an Fc region of an immunoglobulin, wherein the FGF21 mutant protein comprises at least one mutation selected from the group consisting of the mutations (1) to (7) below:
(1) a substitution of amino acids at positions 98 to 101 from the N-terminus of a wild-type FGF21 protein with the amino acid sequence of EIRP (SEQ ID NO: 53);
(2) a substitution of amino acids at positions 170 to 174 from the N-terminus of a wild-type FGF21 protein with the amino acid sequence of TGLEAV (SEQ ID NO: 54);
(3) a substitution of amino acids at positions 170 to 174 from the N-terminus of a wild-type FGF21 protein with the amino acid sequence of TGLEAN (SEQ ID NO: 55);
(4) a substitution of an amino acid at position 170 from the N-terminus of a wild-type FGF21 protein with the amino acid N;
(5) a substitution of an amino acid at position 174 from the N-terminus of a wild-type FGF21 protein with the amino acid N;
(6) a substitution of an amino acid at position 180 from the N-terminus of a wild-type FGF21 protein with the amino acid E, along with one or more mutations (1) to (5) above; and
(7) a mutation of 1 to 10 amino acids for reducing immunogenicity of a wild-type FGF21 protein.

2. The method according to claim 1, wherein the biologically active protein is one selected from the group consisting of insulin, C-peptide, leptin, glucagon, gastrin, gastric inhibitory polypeptide (GIP), amylin, calcitonin, cholecystokinin, peptide YY, neuropeptide Y, bone morphogenetic protein-6 (BMP-6), bone morphogenetic protein-9 (BMP-9), oxyntomodulin, oxytocin, glucagon-like peptide-1 (GLP-1), glucagon-like peptide-2 (GLP-2), irisin, fibronectin type III domain-containing protein 5 (FNDC5), apelin, adiponectin, C1q and tumor necrosis factor related protein (CTRP family), resistin, visfatin, omentin, retinol binding protein-4 (RBP-4), glicentin, angiopoietin, interleukin-22 (IL-22), exendin-4 and growth hormone.

3. The method according to claim 1, wherein the dual function protein comprises the biologically active protein, the Fc region of the immunoglobulin and the FGF21 mutant protein, connected in this order from the N-terminus to the C-terminus of the dual function protein.

4. The method according to claim 1, wherein the dextran sulfate has a weight average molecular weight of 20 to 5,000 kDa.

5. The method according to claim 1, wherein the culture medium contains the dextran sulfate at a concentration of 0.01 to 10 g/L.

6. The method according to claim 1, wherein the culturing comprises:
a step for primary-culturing the mammalian host cell at 34 to 37° C. in a culture medium supplemented with dextran sulfate; and
a step for secondary-culturing the primary-cultured medium at 28 to 33° C.

7. The method of claim 6, wherein the primary-culturing is conducted for 24 to 144 hours.

8. The method of claim 6, wherein the secondary-culturing is conducted at 31 to 33° C.


SEQUENCE LISTING (58 sequences)

SEQ ID NO: 1 - protein, 181 aa, Homo sapiens
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 2 - protein, 30 aa, synthetic construct
  REGION 1..30: note = linker
AKATTAPATTRNTGRGGEEKKKEKEKEEQE

SEQ ID NO: 3 - protein, 17 aa, synthetic construct
  REGION 1..17: note = linker
AKATTAPATTRNTGRGG

SEQ ID NO: 4 - protein, 15 aa, synthetic construct
  REGION 1..15: note = linker
GGGGSGGGGSGGGGS

SEQ ID NO: 5 - protein, 16 aa, synthetic construct
  REGION 1..16: note = linker
GGGGSGGGGSGGGGSA

SEQ ID NO: 6 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVGPSQGRSPSYAS

SEQ ID NO: 7 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVTGLEAVRSPSYAS

SEQ ID NO: 8 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVTGLEANRSPSYAS

SEQ ID NO: 9 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVNPSQGRSPSYAS

SEQ ID NO: 10 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQNRSPSYAS

SEQ ID NO: 11 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 12 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEANRSPSYAS

SEQ ID NO: 13 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVNPSQGRSPSYAS

SEQ ID NO: 14 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVGPSQNRSPSYAS

SEQ ID NO: 15 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVGPSQGRSPSYES

SEQ ID NO: 16 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVTGLEAVRSPSYES

SEQ ID NO: 17 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVTGLEANRSPSYES

SEQ ID NO: 18 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVNPSQGRSPSYES

SEQ ID NO: 19 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQNRSPSYES

SEQ ID NO: 20 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYES

SEQ ID NO: 21 - protein, 182 aa, synthetic construct
  REGION 1..182: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEANRSPSYES

SEQ ID NO: 22 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVNPSQGRSPSYES

SEQ ID NO: 23 - protein, 181 aa, synthetic construct
  REGION 1..181: note = FGF21 variant
HPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVGPSQNRSPSYES

SEQ ID NO: 24 - protein, 229 aa, synthetic construct
  REGION 1..229: note = Human IgG4 Fc
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 228 aa, synthetic construct
  REGION 1..228: note = Human IgG4 Fc variant
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 26 - protein, 223 aa, synthetic construct
  REGION 1..223: note = Hybrid Fc variant
ETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 245 aa, synthetic construct
  REGION 1..245: note = hybrid Fc5
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 233 aa, synthetic construct
  REGION 1..233: note = hybrid Fc40
EKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 31 aa, synthetic construct
  REGION 1..31: note = GLP-1
HAEGTFTSDVSSYLEGQAAKEFIAWLVKGRG

SEQ ID NO: 30 - protein, 31 aa, synthetic construct
  REGION 1..31: note = GLP-1 variant
HGEGTFTSDVSSYLEGQAAKEFIAWLVKGRG

SEQ ID NO: 31 - protein, 31 aa, synthetic construct
  REGION 1..31: note = GLP-1 variant
HGEGTFTSDVSSYLEEQAAKEFIAWLVKGRG

SEQ ID NO: 32 - protein, 31 aa, synthetic construct
  REGION 1..31: note = GLP-1 variant
HGEGTFTSDVSSYLEGQAAKEFIAWLVKGGG

SEQ ID NO: 33 - protein, 31 aa, synthetic construct
  REGION 1..31: note = GLP-1 variant
HGEGTFTSDVSSYLEEQAAKEFIAWLVKGGG

SEQ ID NO: 34 - protein, 276 aa, synthetic construct
  REGION 1..276: note = GLP-1 variant connected to hybrid Fc5
HGEGTFTSDVSSYLEGQAAKEFIAWLVKGRGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 264 aa, synthetic construct
  REGION 1..264: note = GLP-1 variant connected to hybrid Fc40
HGEGTFTSDVSSYLEGQAAKEFIAWLVKGRGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 276 aa, synthetic construct
  REGION 1..276: note = GLP-1 variant connected to hybrid Fc5
HGEGTFTSDVSSYLEEQAAKEFIAWLVKGRGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 264 aa, synthetic construct
  REGION 1..264: note = GLP-1 variant connected to hybrid Fc40
HGEGTFTSDVSSYLEEQAAKEFIAWLVKGRGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 276 aa, synthetic construct
  REGION 1..276: note = GLP-1 variant connected to hybrid Fc5
HGEGTFTSDVSSYLEGQAAKEFIAWLVKGGGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 264 aa, synthetic construct
  REGION 1..264: note = GLP-1 variant connected to hybrid Fc40
HGEGTFTSDVSSYLEGQAAKEFIAWLVKGGGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 276 aa, synthetic construct
  REGION 1..276: note = GLP-1 variant connected to hybrid Fc5
HGEGTFTSDVSSYLEEQAAKEFIAWLVKGGGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 264 aa, synthetic construct
  REGION 1..264: note = GLP-1 variant connected to hybrid Fc40
HGEGTFTSDVSSYLEEQAAKEFIAWLVKGGGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 275 aa, synthetic construct
  REGION 1..275: note = Dulaglutide
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 43 - protein, 461 aa, synthetic construct
  REGION 1..461: note = GLP1(A2G)-HyFc40-GS3-FGF21(EIRP, TGLEAV)
HGEGTFTSDVSSYLEGQAAKEFIAWLVKGRGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 44 - protein, 473 aa, synthetic construct
  REGION 1..473: note = GLP1(GE)-HyFc5-GS3-FGF21(EIRP, TGLEAV)
HGEGTFTSDVSSYLEEQAAKEFIAWLVKGRGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 45 - protein, 461 aa, synthetic construct
  REGION 1..461: note = GLP1(GE)-HyFc40-GS3-FGF21(EIRP, TGLEAV)
HGEGTFTSDVSSYLEEQAAKEFIAWLVKGRGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 46 - protein, 473 aa, synthetic construct
  REGION 1..473: note = GLP1(GG)-HyFc5-GS3-FGF21(EIRP, TGLEAV)
HGEGTFTSDVSSYLEGQAAKEFIAWLVKGGGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 47 - protein, 461 aa, synthetic construct
  REGION 1..461: note = GLP1(GG)-HyFc40-GS3-FGF21(EIRP, TGLEAV)
HGEGTFTSDVSSYLEGQAAKEFIAWLVKGGGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 48 - protein, 473 aa, synthetic construct
  REGION 1..473: note = GLP1(GEG)-HyFc5-GS3-FGF21(EIRP, TGLEAV)
HGEGTFTSDVSSYLEEQAAKEFIAWLVKGGGRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 49 - protein, 461 aa, synthetic construct
  REGION 1..461: note = GLP1(GEG)-HyFc40-GS3-FGF21(EIRP, TGLEAV)
HGEGTFTSDVSSYLEEQAAKEFIAWLVKGGGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYAS

SEQ ID NO: 50 - protein, 461 aa, synthetic construct
  REGION 1..461: note = GLP1(GEG)-HyFc40-GS3-FGF21(EIRP, TGLEAV, A180E)
HGEGTFTSDVSSYLEEQAAKEFIAWLVKGGGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEAVRSPSYES

SEQ ID NO: 51 - protein, 461 aa, synthetic construct
  REGION 1..461: note = GLP1(GEG)-HyFc40-GS3-FGF21(EIRP, TGLEAN, A180E)
HGEGTFTSDVSSYLEEQAAKEFIAWLVKGGGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVTGLEANRSPSYES

SEQ ID NO: 52 - protein, 460 aa, synthetic construct
  REGION 1..460: note = GLP1(GEG)-HyFc40-GS3-FGF21(EIRP, G170N, A180E)
HGEGTFTSDVSSYLEEQAAKEFIAWLVKGGGEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFREEIRPDGYNVYQSEAHGLPLHLPGNKSPHRDPAPRGPARFLPLPGLPPALPEPPGILAPQPPDVGSSDPLSMVNPSQGRSPSYES

SEQ ID NO: 53 - protein, 4 aa, synthetic construct
  REGION 1..4: note = FGF21 variant
EIRP

SEQ ID NO: 54 - protein, 6 aa, synthetic construct
  REGION 1..6: note = FGF21 variant
TGLEAV

SEQ ID NO: 55 - protein, 6 aa, synthetic construct
  REGION 1..6: note = FGF21 variant
TGLEAN

SEQ ID NO: 56 - protein, 25 aa, synthetic construct
  REGION 1..25: note = derived sequences
MDAMLRGLCCVLLLCGAVFVSPSHA

SEQ ID NO: 57 - protein, 4 aa, synthetic construct
  REGION 1..4: note = synthetic construct
LLLE

SEQ ID NO: 58 - protein, 5 aa, synthetic construct
  REGION 1..5: note = Synthetic construct
GPSQG